(19) United States
(12) Patent Application Publication
(10) Pub. No.: US 2004/0059384 A1
(43) Pub. Date: Mar. 25, 2004

(54) Lower frequency health assistor

(76) Inventor: Tsung-I Yu, Pan Chiao City (TW)

Correspondence Address: BACON & THOMAS, PLLC, 625 SLATERS LANE, FOURTH FLOOR, ALEXANDRIA, VA, 22314

(21) Appl. No.: 10/253,817

(22) Filed: Sep. 25, 2002

(51) Int. Cl. A61N 1/00

(52) U.S. Cl. 607/2

(57) ABSTRACT

A lower frequency health assistor comprising a conductive plastic film, a film adhesive object and a controller. A conductive plastic film is a thin piece and has at least one pair of conductive surfaces of different polarities which are opened. A film adhesive object for installing the conductive plastic film; and capable of being worn on human body. A controller for switching and adjusting the current and having a variety of functions. The conductive plastic film is installed to a predetermined position at an inner surface of the film adhesive object so that the conductive plastic film contacts the body; the controller actuates a lower frequency vibration to the human body.

### BACKGROUND OF THE INVENTION

(a) Field of the Invention

The present invention relates to a lower frequency health assistor formed by a conductive plastic film, a film adhesive object and a controller. The controller serves to switch and adjust the current and has a variety of functions. The conductive plastic film has at least one pair of conductive surfaces and can be adhered to the human body so as to massage the acupuncture point of human body by generating lower frequency vibration.

(b) Description of the Prior Art:

The prior art lower frequency health assistor (for example, massagers, or treater) includes mainly a controller, a silicide electrode pieces or silicide soft glue (called as conductive glue), and bowl shape silicon electrode, etc. The manufacturing cost is high and the timing or place for use is greatly confined. Moreover, the conductivity of the prior art lower frequency health assistor is not preferred. Especially, the user must be coated with silicon glue at the portion to contact the lower frequency health assistor. The glue causes that the user feel uneasy. Besides, the silicide soft glue, and bowl shape silicon electrode are heavy so that the user will be uncomfortable and the lower frequency health assistor can not be hidden well.

Referring to FIGS. 23 and 24, a prior art silicon electrode cups 10 have two conductive silicon of different polarities so that the controller is connected to two polarities of a conductive silicon 20. In use, the conductive glue 30 (silicon glue) is coated on the inner walls of the conductive silicon 20 of the cups 20 so that the conductive glue 20 can adhere to the acupuncture point 41 of human body (referring to FIG. 22). Then low current flows through the body 40. However, the user will feel uneasy and feel heavy as it is worn on the body.

### SUMMARY OF THE INVENTION

Accordingly, the primary object of the present invention is to provide a lower frequency health assistor, wherein The conductive plastic film of the present invention is a soft thin piece so as to have a preferred contact to the human skin so that it is suitable for different film adhesive objects (such as brassier, strips or trousers, etc.). The conductive plastic film has a good conductivity, is light-weighted and is a hidden structure with a less space. Moreover, the conductivity will not be destroyed by watch or folding.

Another object of the present invention is to provide a lower frequency health assistor, wherein the conductive plastic film of the present invention is The conductive plastic film is formed by a plastic PC board with a printed circuit board so as to form with conductive surfaces with different polarities and conductive paths. It has a simple structure and is made with a low cost. Moreover, the area of the conductive plastic film is smaller, for example, an area of about 1 center meter square.

A third object of the present invention is to provide a lower frequency health assistor, wherein by the lower frequency vibration, heat is generated in 15 to 30 minutes so as to improve circulation of blood so as to enhance the physical function of human body.

A fourth object of the present invention is to provide a lower frequency health assistor, wherein when the present invention is used to the breast of female, the conductive plastic film has a smaller volume, and thus the controller can be hidden in between the two cups, or the controller is placed out of the body so as to have a beautiful outlook.

A fifth object of the present invention is to provide a lower frequency health assistor, wherein the present invention can be used to male or female trousers, it is formed with though hole 21C so that it will not affect as in making love.

A sixth object of the present invention is to provide a lower frequency health assistor, wherein the conductive plastic film is a thin piece and thus can be adhered to the body. Thereby, the tips are positioned with respect to the acupuncture point so as to have the function of massage.

A seventh object of the present invention is to provide a lower frequency health assistor, wherein the controller has a clip, and thereby, it can be carried conveniently and the stageless current control causes that the user can adjust the current as desir

To achieve above object, the present invention provides a lower frequency health assistor comprising a conductive plastic film, a film adhesive object and a controller. The conductive plastic film is a thin piece and has at least one pair of conductive surfaces of different polarities which are opened; a film adhesive object for installing the conductive plastic film; and capable of being worn on human body; and a controller for switching and adjusting the current and having a variety of functions. The conductive plastic film is installed to a predetermined position at an inner surface of the film adhesive object so that the conductive plastic film contacts the body; the controller actuates a lower frequency vibration to the human body.

### BRIEF DESCRIPTION OF THE DRAWINGS

FIG. 1 is a partial schematic view showing the assembly of the conductive plastic film of the present invention.

FIG. 2 is a cross section view of the conductive plastic film of the present invention.

FIG. 3 is a schematic view includes the brassier type film adhesive object of the present invention.

FIG. 4 is a schematic view showing the clothes type film adhesive object of the present invention.

FIG. 5 is a schematic view showing the integral formed film adhesive object of the present invention.

FIG. 6 is a schematic view showing the male trousers film adhesive object according to the present invention.

FIG. 7 is a schematic view showing the female film adhesive object of the present invention.

FIG. 8 is a schematic view showing that the present invention is used in a trouser film adhesive object.

FIG. 9 is a schematic view showing that the present invention is used in a trouser film adhesive object.

FIG. 10 is a schematic view showing that the conductive plastic film is formed with tips in the present invention.

FIG. 11 is a schematic view showing that the tips of the conductive plastic film are used to extrude the hair in the body.

FIG. 12 is a schematic view showing that the trouser of the present invention is worn on the body.

FIG. 13 shows one application that the present invention is used to a strip conductive plastic film.

FIG. 14 is another application where the present invention is used to a strip conductive plastic film.

FIG. 15 is a further application showing that the present invention is used to a ring conductive plastic film.

FIG. 16 is another schematic view showing that the present invention is used to a ring conductive plastic film.

FIG. 17 shows one application that the controller of the present invention is used as a stageless switch.

FIGS. 18 to 21 shows the different configurations of the conductive surfaces of the present invention.

FIG. 22 is a schematic view showing the acupuncture point of a human body.

FIG. 23 is a schematic perspective view of a prior art conductive cups of a brassier.

FIG. 24 is a cross section view includes of a prior art conductive cups of a brassier.

### DETAILED DESCRIPTION OF THE PREFERRED EMBODIMENTS

Referring to drawings, the lower frequency health assistor of the present invention is illustrated. The lower frequency health assistor mainly includes a conductive plastic film 1, a film adhesive object, and a controller.

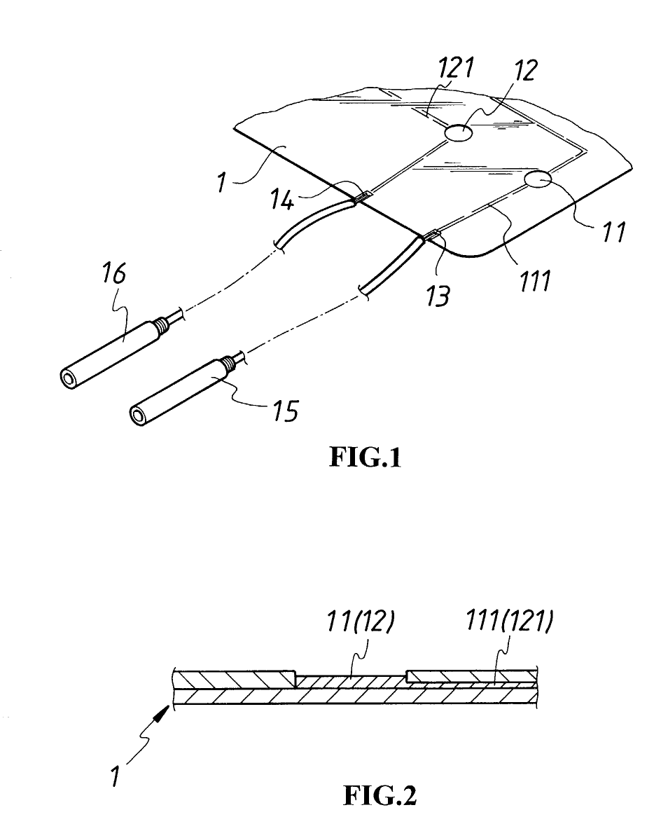

The conductive plastic film 1, referring to FIGS. 1 and 2, is a soft and thin film. The conductive plastic film is formed by a plastic PC board with a printed circuit board so as to form with conductive surfaces 11 and 12 with different polarities and conductive paths 111 and 121. The conductive plastic film 1 may have different shape and configuration according to the portion of a body. A predetermined position on a surface of the thin film has the set of the conductive surfaces 11 and 12 with different polarities. Each of the conductive surfaces 11 and 12 is connected to one of electrode joints 13 and 14 through the conductive paths 111 and 121. Each joint 13, 14 is connected to a conductive inserting pin 15, 16. The conductive paths 111 and 121 may be installed in a thin clamping layer of the conductive plastic film 1, and only the conductive surfaces 11 and 12 exposes out. The conductive surfaces 11 and 12 of different polarities are open-circuited.

The film adhesive object is a soft object. The conductive plastic film 1 can be installed on the film adhesive object and the film adhesive object can be worn on a predetermined portion of a body. The shape of the conductive plastic film 1 can be embodied by the desired shape and structure. The embodiments of the film adhesive object will be described herein.

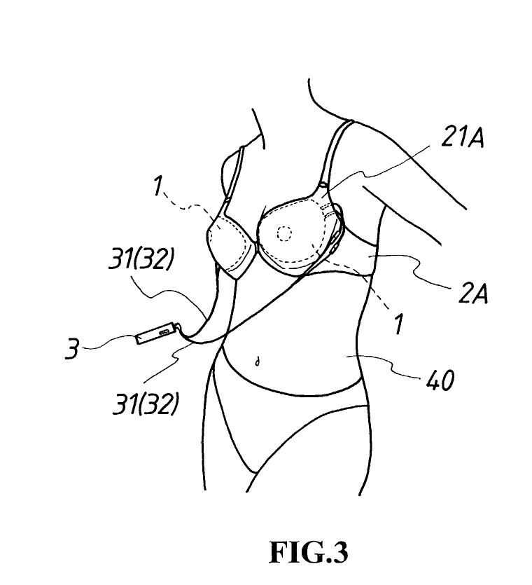

1. Referring to FIG. 3, the film adhesive object has a structure of brassiere 2A. The inner part of the cup 21A of the brassiere 2A is adhered with the conductive plastic film 1.

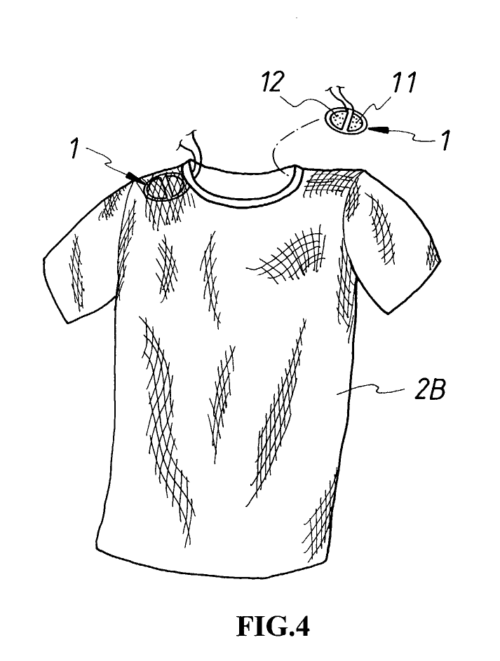

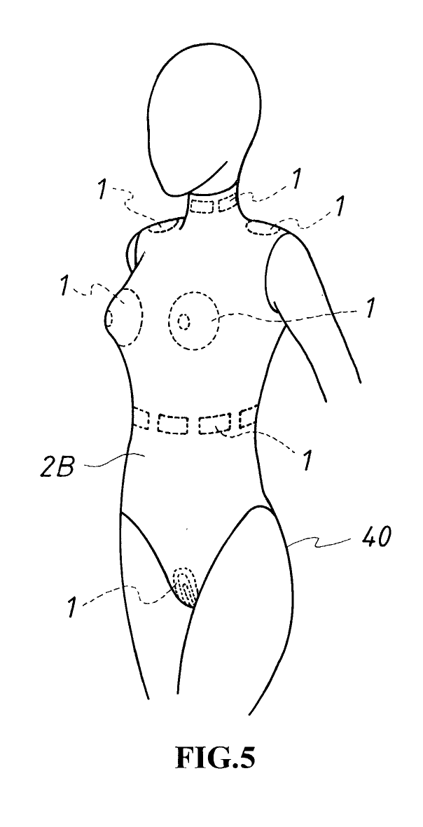

2. Referring to FIG. 4, the film adhesive object is an elastic net 2B. The inner part of the shoulder of a predetermined clothes 2B is adhered with the conductive plastic film 1. The inner part of clothes 2B is installed with the conductive plastic film 1 at a position with respect to the neck, shoulder, breast, waist, etc. (referring to FIG. 5).

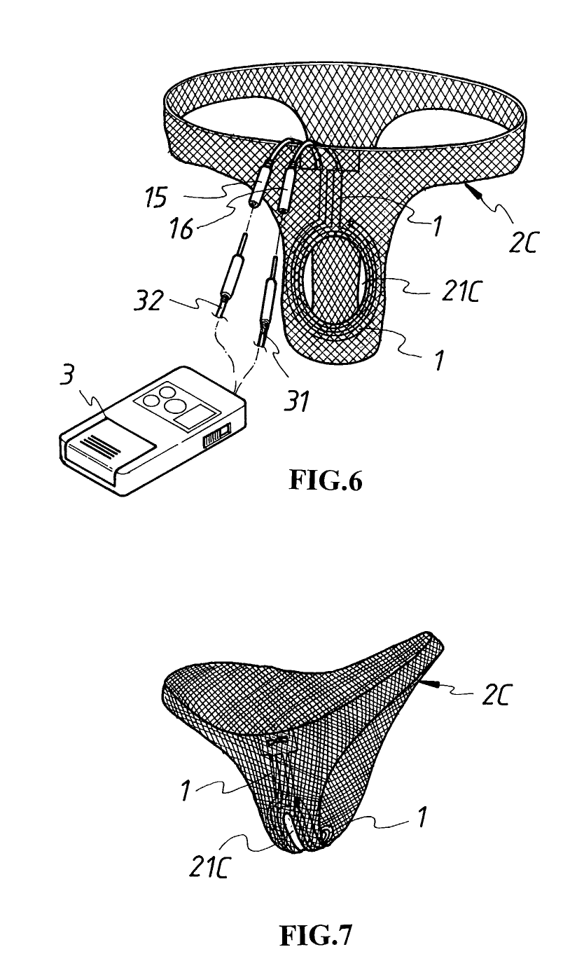

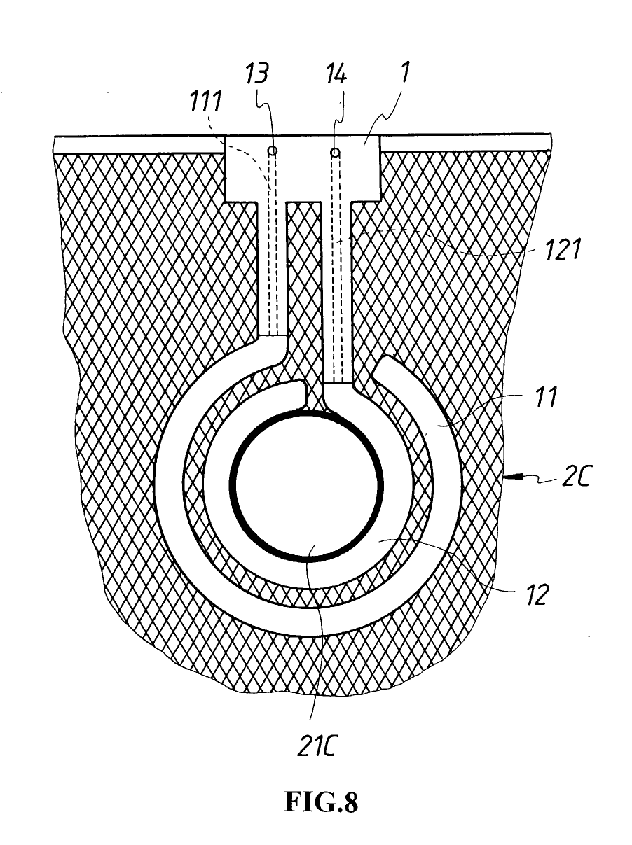

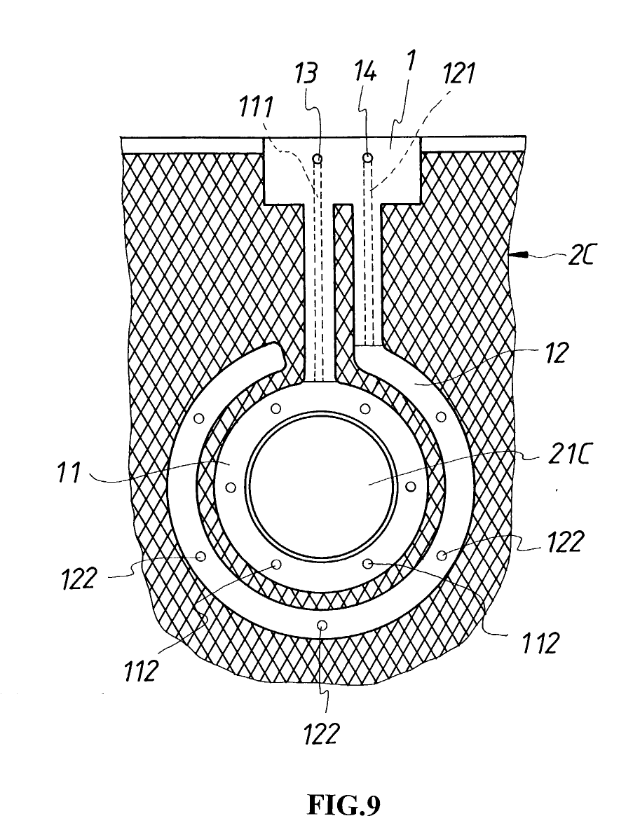

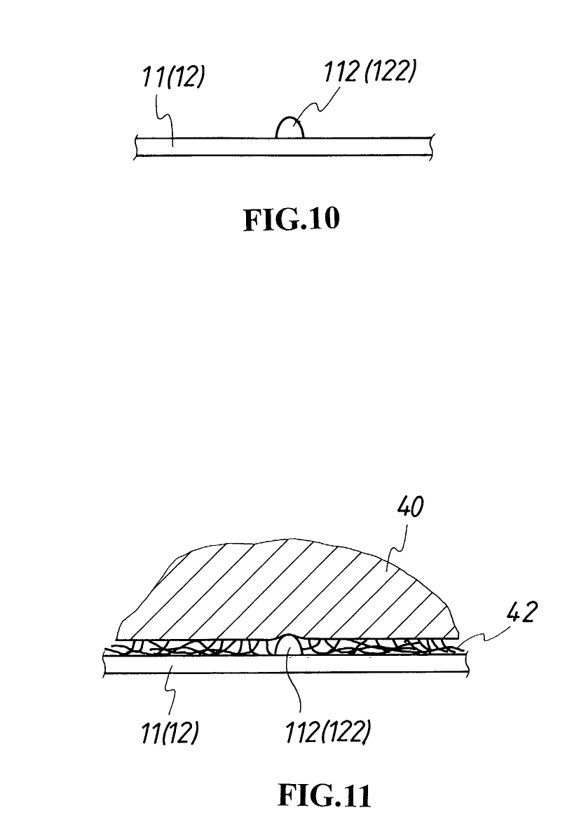

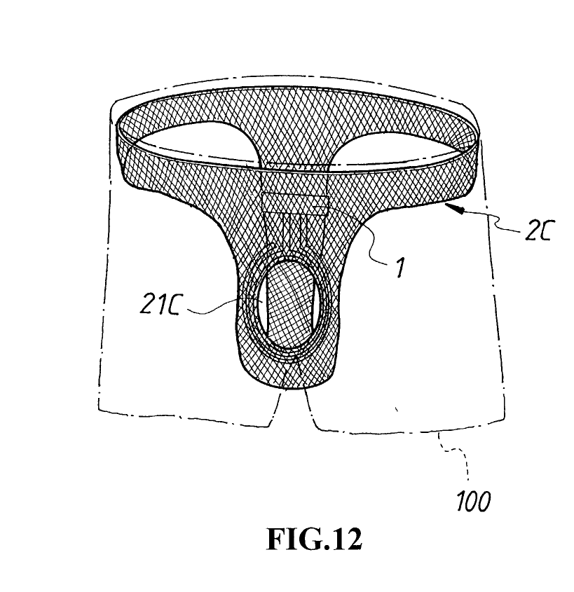

3. Referring to FIGS. 6 and 7, the film adhesive object is a male or female trouser 2C. The physiological portion of the trouser 21 is formed with a though hole 21C. A periphery of the though hole 21C is installed with an annular conductive plastic film 1 (referring to FIGS. 8 and 9). The conductive surfaces 11 and 12 of the conductive plastic film 1 are formed with a plurality of conductive tips 112, 122, especially, those illustrated in FIG. 10. Each of pits 112, 122 is a proper protruding particle for extruding the hairs of human body so as to contact the body (referring to FIG. 11). Thereby, it can be adhered to the trousers 100 of the user, see FIG. 12.

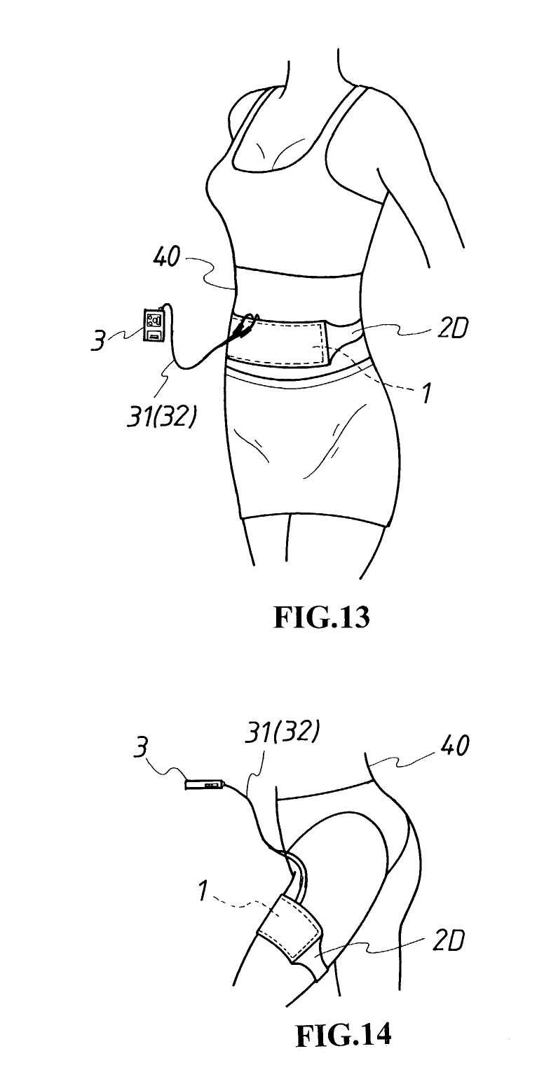

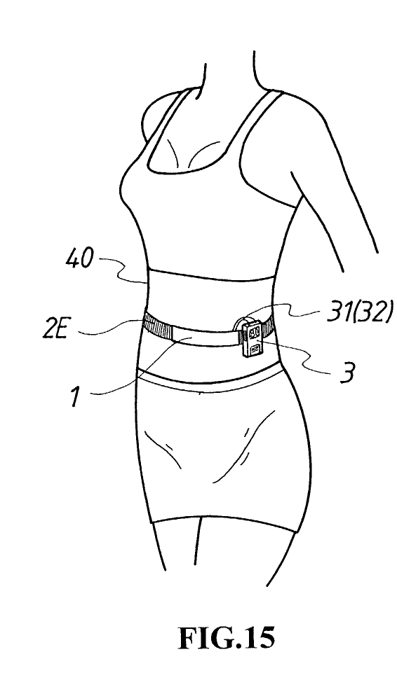

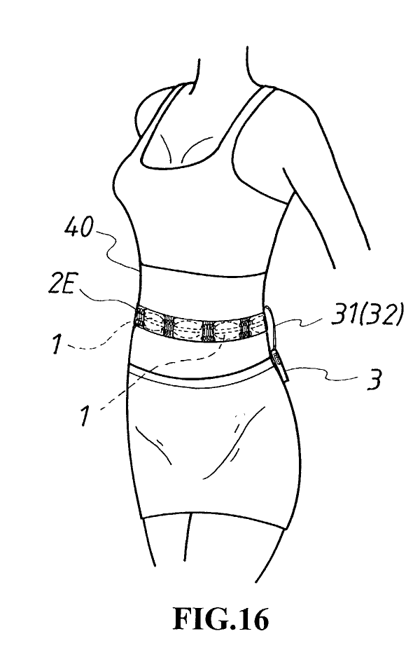

4. Referring to FIGS. 13 to 16, the film adhesive object is a strip 2D or 2E which may be a sticky strip and may be connected with one another. One or a plurality of portions of the strip are installed with the conductive plastic films 1 for adhering to the body of the user.

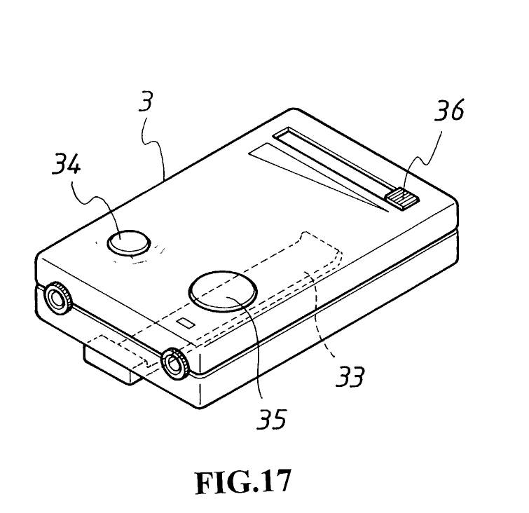

Referring to FIGS. 3 to 6, the controller serves for switching or adjusting the current. The interior of the controller has a battery. Two conductive wires 31, 32 with different polarities are connected to the conductive pins 15, 16 of the conductive plastic film 1. Referring to FIG. 8, the controller may have a clip 33 for fixing to the user. The control switches 34, 35 are used in switching and a switch 36 serves to control the strength of current. The switch is a pull type or a stageless switch (the controller in FIG. 17 is a pull stageless switch).

By above components, the conductive plastic film 1 can be installed to the inner surface of the predetermined portion of different film adhesive objects 2A to 2E so that the conductive surfaces 11 and 12 exactly adheres to the physiologic portion or acupuncture point portion 41 or the muscle (referring to FIG. 17, the acupuncture point of human body is illustrated) so that when the controller 3 is actuated, lower current generates lower frequency vibration to massage human body 40, acupuncture point 41, or muscle or other physiological portion, etc.

The conductive plastic film 1 of the present invention is a light thin piece, which can be adhered to the film adhesive objects 2A to 2E and adhered to the skin of body 40. The contact is unnecessary to coat any conductive glue. After use, the user does not need to flush body. Thereby, it is portable easily and conveniently.

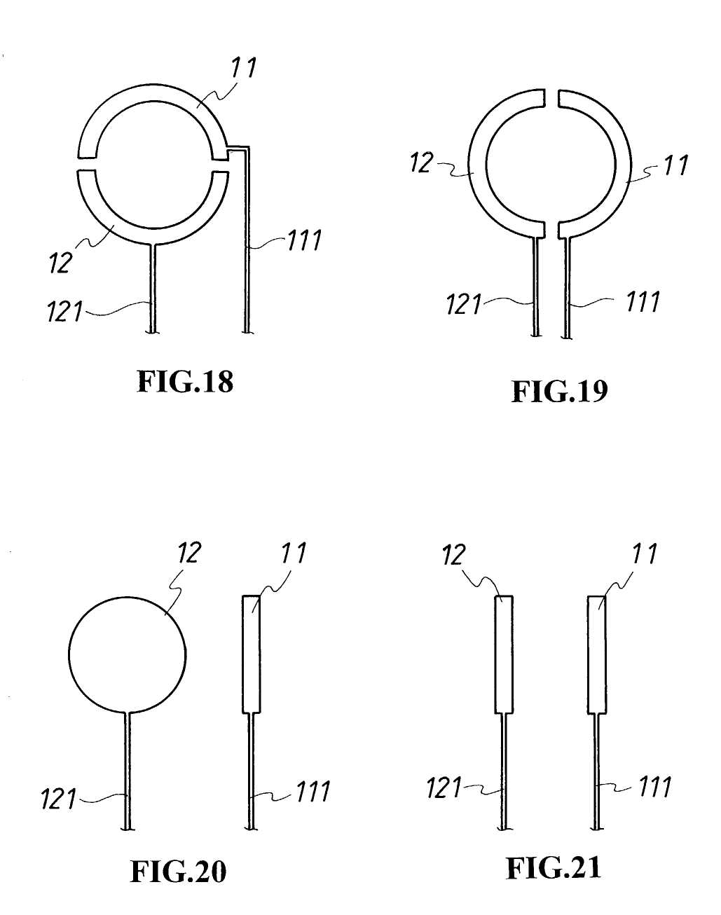

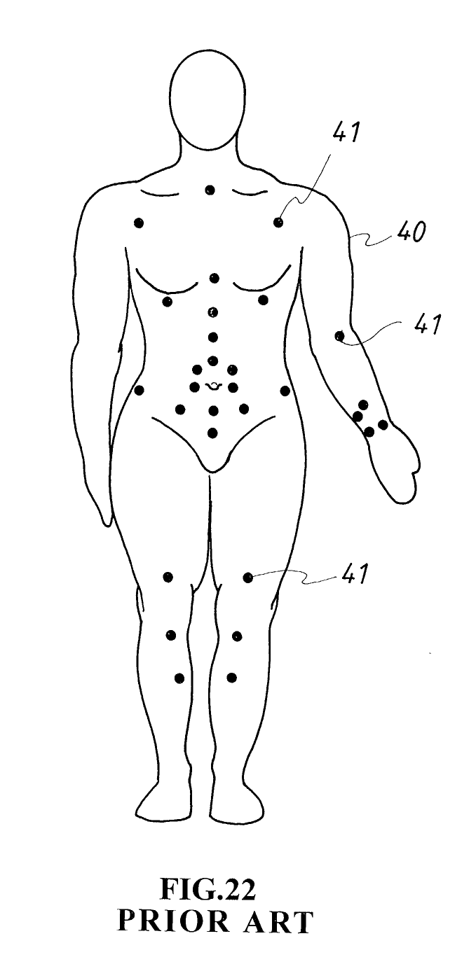

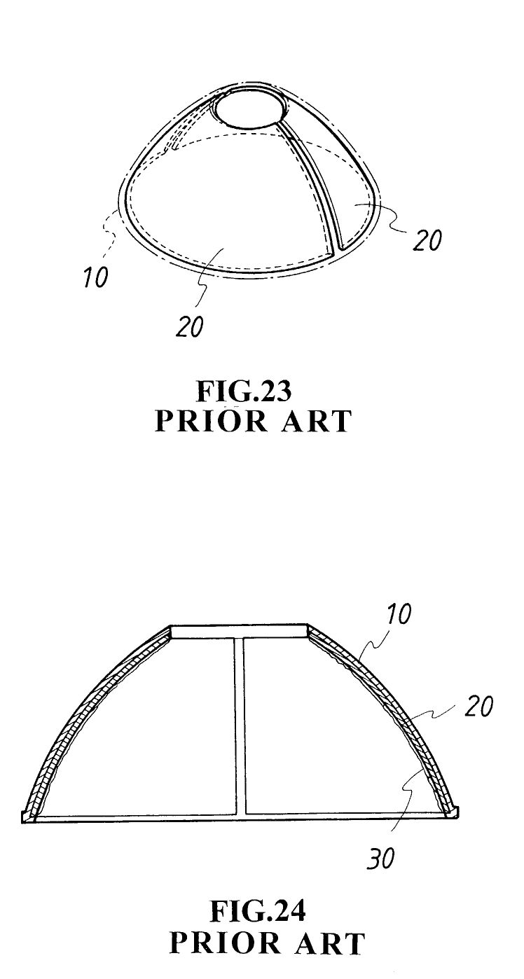

The conductive plastic film 1 can be made by printed circuit board massively. The configurations of the conductive surfaces 11 and 12, conductive paths 111 and 121 are dependent on the body to be contacted. Referring to FIGS. 18 to 21, the conductive surfaces 11 and 12 may have cambered shapes, round shape or long shapes. The conductive paths 111 and 121 can be connected to electrodes. If the present invention is used to acupuncture point of human body, a plurality of conductive surfaces 11 and 12 can be adhered to the contact portions so as to perform a lower frequency vibration to massage bodies.

The described conductive plastic film 1 and film adhesive objects are not confined to above said embodiments. If necessary, the conductive plastic film 1 can be coated with foam or other soft material for matching different film adhesive objects or the conductive plastic film is directly adhered to the inner surface of the film adhesive object.

1. The conductive plastic film of the present invention is a soft thin piece so as to have a preferred contact to the human skin so that it is suitable for different film adhesive objects (such as brassier, strips or trousers, etc.). The conductive plastic film has a good conductivity, is light-weighted and is a hidden structure with a less space. Moreover, the conductivity will not be destroyed by watch or folding.

2. The conductive plastic film of the present invention is The conductive plastic film is formed by a plastic PC board with a printed circuit board so as to form with conductive surfaces with different polarities and conductive paths. It has a simple structure and is made with a low cost. Moreover, the area of the conductive plastic film is smaller, for example, an area of about 1 center meter square.

3. By the lower frequency vibration, heat is generated in 15 to 30 minutes so as to improve circulation of blood so as to enhance the physical function of human body.

4. When the present invention is used to the breast of female, the conductive plastic film has a smaller volume, and thus the controller can be hidden in between the two cups, or the controller is placed out of the body so as to have a beautiful outlook.

5. Referring to FIGS. 6 and 7, the present invention can be used to male or female trousers, it is formed with though hole 21C so that it will not affect as in making love.

6. The conductive plastic film is a thin piece and thus can be adhered to the body. Thereby, the tips are positioned with respect to the acupuncture point so as to have the function of massage.

7. The controller has a clip, and thereby, it can be carried conveniently and the stageless current control causes that the user can adjust the current as desired.

Although the present invention has been described with reference to the preferred embodiments, it will be understood that the invention is not limited to the details described thereof. Various substitutions and modifications have been suggested in the foregoing description, and others will occur to those of ordinary skill in the art. Therefore, all such substitutions and modifications are intended to be embraced within the scope of the invention as defined in the appended claims.

1. A lower frequency health assistor comprising a conductive plastic film, a film adhesive object and a controller; characterized in that:
a conductive plastic film is a thin piece and has at least one pair of conductive surfaces of different polarities which are open-circuited;
a film adhesive object for installing the conductive plastic film; and capable of being worn on human body; and
a controller for switching and adjusting the current and having a variety of functions;
wherein the conductive plastic film is installed to a predetermined position at an inner surface of the film adhesive object so that the conductive plastic film contacts the body;
the controller actuates a lower frequency vibration to the human body.

2. The lower frequency health assistor as claim in claim 1, wherein the conductive surfaces of the conductive plastic film have respective conductive paths which are connected to respective polarity joints, each polarity joints is connected to a conductive inserting pin.

3. The lower frequency health assistor as claim in claim 1, wherein the conductive plastic film is formed by a plastic PC board with a printed circuit board so as to form with conductive surfaces with different polarities and conductive paths.

4. The lower frequency health assistor as claim in claim 1, wherein the conductive path is connected to a polarity joint and is installed in a clamping layer of the conductive plastic film.

5. The lower frequency health assistor as claim in claim 1, wherein the conductive plastic film is selected from one of brassier, clothes, trousers, strips, and rings.

6. The lower frequency health assistor, wherein the controller has two conductive wires of different polarities which are connected to the conductive surfaces, respectively.

7. The lower frequency health assistor as claim in claim 1, 2 or 3, wherein the conductive surfaces are installed with tips.

8. The lower frequency health assistor as claim in claim 1, wherein an outer side of the conductive plastic film is coated with foams or soft materials.

9. The lower frequency health assistor as claim in claim 1, wherein the conductive plastic film is adhered or combined to an inner wall of a predetermined portion of the film adhesive object.

10. The lower frequency health assistor as claim in claim 1, wherein the controller is installed with a clip.

11. The lower frequency health assistor as claim in claim 1, wherein the controller is a stageless switch for adjusting the strength of a current.
